(19) United States
(12) Patent Application Publication
(10) Pub. No.: US 2005/0137416 A1
(43) Pub. Date: Jun. 23, 2005

(54) Process and intermediates for preparing a cyclohexylnitrile

(75) Inventor: Kevin Scott Webb, Port St. Lucie, FL (US)

Correspondence Address: GLAXOSMITHKLINE;Corporate Intellectual Property - UW2220, P.O. Box 1539, King of Prussia, PA, 19406-0939, US

(73) Assignee: SmithKline Beecham Corporation

(21) Appl. No.: 11/017,081

(22) Filed: Dec. 20, 2004

Related U.S. Application Data
(63) Continuation of application No. 10/181,039, filed on Jul. 11, 2002, now abandoned.
(60) Provisional application No. 60/175,720, filed on Jan. 12, 2000.

(51) Int. Cl. C07C 253/30

(52) U.S. Cl. 558/410

(57) ABSTRACT

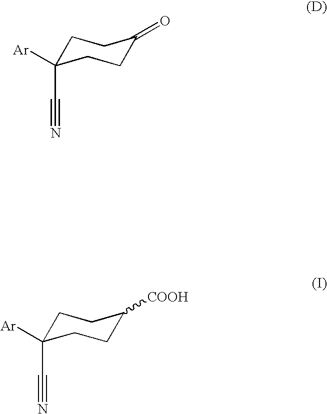

Cyanohydrin homologation of the 4-cyanocyclohexanone (D) provides a 4-cyanocyclohexanoic acid (I).

### AREA OF THE INVENTION

This invention relates to a method and intermediates for preparing certain nitriles which are useful for making cyclohexanoic acids. The latter are pharmaceutically active agents.

### BACKGROUND OF THE INVENTION

The process and intermediates of this invention provide a means for making certain 4-substituted-4-(3,4-disubstituted phenyl)cyclohexanoic acids which are useful for treating asthma and other diseases which can be moderated by affecting the PDE 4 enzyme. The final products of particular interest are fully described in U.S. Pat. No. 5,552,438 issued Sep. 3, 1996. The information and representations disclosed therein, in so far as that information and those representations are necessary to the understanding of this invention and its practice, are incorporated herein by reference, in total.

This invention discloses a method for preparing a cyclohexanoic acid by cyanohydrin homologation of a cyclohexanone precursor.

### SUMMARY OF THE INVENTION

A process for preparing a compound of formula (I) 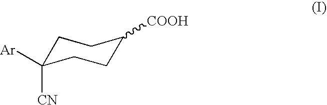
where Ar is an aromatic group,

wherein the process comprises reducing a α,β-unsaturated cyclohexene carboxylic acid of formula (A).

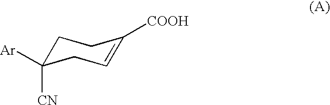

In as second aspect, this invention relates to a process for preparing an α,β-unsaturated cyclohexene carboxylic acid of formula (A) 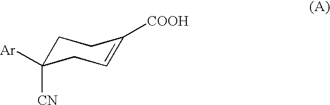
which process comprises hydrolysing a compound of formula (B).

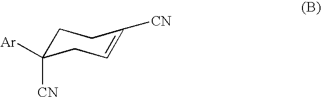

In yet a further aspect, this invention relates to a process for preparing a compound of formula (I) by the cyanohydrin homologation of cyclohexanone (X) as described herein 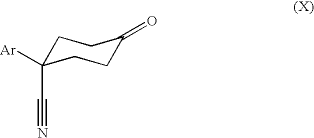
wherein Ar is an aromatic group.

### DESCRIPTION OF THE INVENTION

This invention can be used to homologate any cyclohexanone. In this invention the cyclohexanone has a 4-position nitrile group with or without another group at that 4 position. Since the homologation is believed to be independent of the 4-position substitutent(s), the scope of the compounds which are homologated herein are simply examples of the chemistries being done on the 1-position carbon. Herein the invention is exemplified by compounds which have an aromatic group at the 4 position in addition to the nitrile group.

A preferred group of compounds which can be prepared by this homologation route are those of formula (Ia) 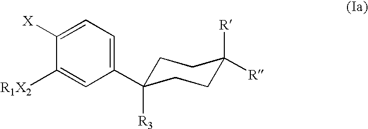
wherein
R₁ is —(CR₄R₅)ᵣR₆ wherein the alkyl moieties may be optionally substituted with one or more halogens;
R₂ is —CH₃ or —CH₂CH₃ optionally substituted by 1 or more halogens;
R₃ is —CN;
r is 0 to 6;
R₄ and R₅ are independently selected from hydrogen or a C₁₋₂ alkyl;
R₆ is hydrogen, methyl, hydroxyl, aryl, halo substituted aryl, aryloxyC₁₋₃ alkyl, halo substituted aryloxyC₁₋₃ alkyl, indanyl, indenyl, C₇₋₁₁ polycycloalkyl, tetrahydrofuranyl, furanyl, tetrahydropyranyl, pyranyl, tetrahydrothienyl, thienyl, tetrahydrothiopyranyl, thiopyranyl, C₃₋₆ cycloalkyl, or a C₄₋₆ cycloalkyl containing one or two unsaturated bonds, wherein the cycloalkyl and heterocyclic moieties may be optionally substituted by 1 to 3 methyl groups or one ethyl group;
R₇ is hydrogen or C₁₋₆ alkyl;
X is YR₂;
X₂ is O or NR₇;
Y is O or S(O)ₘ where m is 0, 1 or 2; and
one of R′ or R″ is hydrogen and the other is COOH or a salt thereof.

Preferred X groups for formula (Ia) are those wherein Y is oxygen. The preferred X₂ group for formula (Ia) is that wherein X₂ is oxygen. Preferred R₂ groups are a C₁₋₂ alkyl unsubstituted or substituted by 1 or more halogens. The halogen atoms are preferably fluorine and chlorine, more preferably fluorine. More preferred R₂ groups are those wherein R₂ is methyl, or the fluoro-substituted alkyls, specifically a C₁₋₂ alkyl, such as a —CF₃, —CHF₂, or —CH₂CHF₂ moiety. Most preferred are the —CHF₂ and —CH₃ moieties.

Most preferred are those compounds wherein R₁ is —CH₂-cyclopropyl, cyclopentyl, 3-hydroxycyclopentyl, methyl or CF₂H; X is YR₂; Y is oxygen; X₂ is oxygen; and R₂ is CF₂H or methyl. The most preferred compounds are cis and trans 4-cyano-4-(3-cyclopentyloxy-4-methoxyphenyl)cyclohexanoic acid, in particular the cis isomer, which is the equitorial form the acid.

As regards the preferred embodiments for formula (A), they are those wherein:
R₁ is —(CR₄R₅)ᵣR₆ wherein the alkyl moieties may be optionally substituted with one or more halogens;
R₂ is —CH₃ or —CH₂CH₃ optionally substituted by 1 or more halogens;
R₃ is —CN;
r is 0 to 6;
R₄ and R₅ are independently selected from hydrogen or a C₁₋₂ alkyl;
R₆ is hydrogen, methyl, hydroxyl, aryl, halo substituted aryl, aryloxyC₁₋₃ alkyl, halo substituted aryloxyC₁₋₃ alkyl, indanyl, indenyl, C₇₋₁₁ polycycloalkyl, tetrahydrofuranyl, furanyl, tetrahydropyranyl, pyranyl, tetrahydrothienyl, thienyl, tetrahydrothiopyranyl, thiopyranyl, C₃₋₆ cycloalkyl, or a C₄₋₆ cycloalkyl containing one or two unsaturated bonds, wherein the cycloalkyl and heterocyclic moieties may be optionally substituted by 1 to 3 methyl groups or one ethyl group;
R₇ is hydrogen or C₁₋₆ alkyl;
X is YR₂; and
X₂ is O or NR₇;
Y is O or S(O)ₘ where m is 0, 1 or 2.

Preferred X groups for formula (A) are those wherein Y is oxygen. The preferred X₂ group for formula (A) is that wherein X₂ is oxygen. Preferred R₂ groups are a C₁₋₂ alkyl unsubstituted or substituted by 1 or more halogens. The halogen atoms are preferably fluorine and chlorine, more preferably fluorine. More preferred R₂ groups are those wherein R₂ is methyl, or the fluoro-substituted alkyls, specifically a C₁₋₂ alkyl, such as a —CF₃, —CHF₂, or —CH₂CHF₂ moiety. Most preferred are the —CHF₂ and —CH₃ moieties.

Most preferred are those compounds wherein R₁ is —CH₂-cyclopropyl, cyclopentyl, 3-hydroxycyclopentyl, methyl or CF₂H; X is YR₂; Y is oxygen; X₂ is oxygen; and R₂ is CF₂H or methyl.

The homologation of substituted cyclohexan-1-ones to the corresponding cyclohexanoic acids are useful in making the compounds of U.S. Pat. No. 5,552,483. With reference to Scheme 1, one route is to convert 4-cyano-4-(3-cyclopentyloxy-4-methoxyphenyl)cyclohexan-1-one (the ketone) to the cyanohydrin of formula 2, then to the α,β-unsaturated nitrile 3, thereafter to the saturated nitrile, and finally to the saturated acid 5 or 6, i.e., sequence 1 [symbol] 2 [symbol] 3 [symbol] 7 [symbol] 5 [symbol] 6. The key step in this sequence is hydrolysis of the saturated nitrile 7 to the saturated acid 5 and 6.

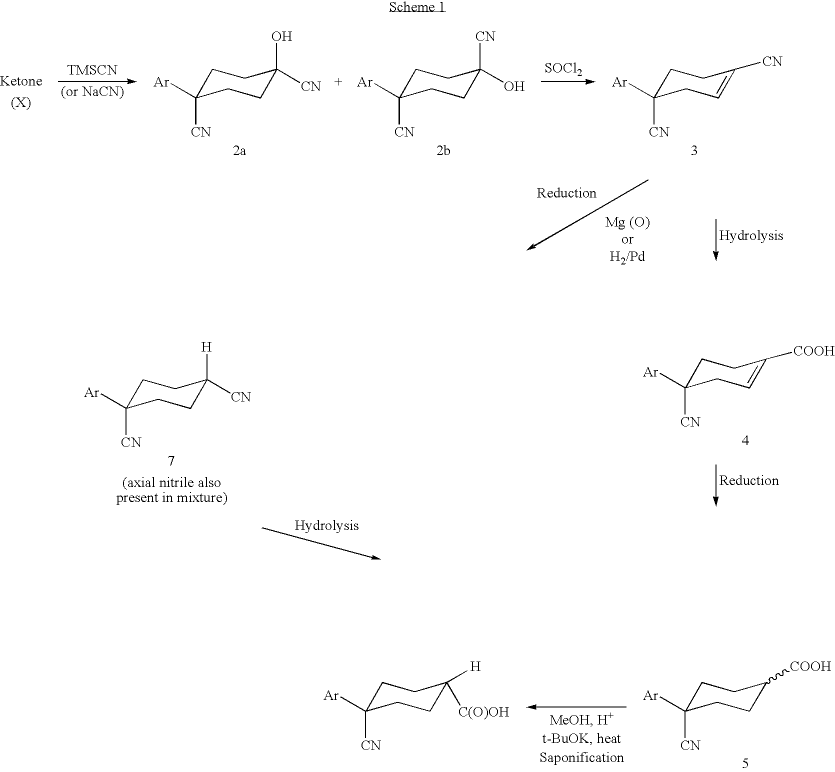

An alternative novel reaction sequence is the one in Scheme 1 illustrated by steps 1 [symbol] 2 [symbol] 3 [symbol] 4 [symbol] 5 [symbol] 6. Intermediate 4 in this sequence, the α,β-unsaturated carboxylate is reduced with Pd/C/cyclohexene and ammonium bicarbonate or via catalytic hydrogenation to yield a mixture of cis and trans isomers.

This invention can be applied generally to the preparation and reduction of an α,β-unsaturated cyclohexene carboxylate as illustrated in Scheme 2. 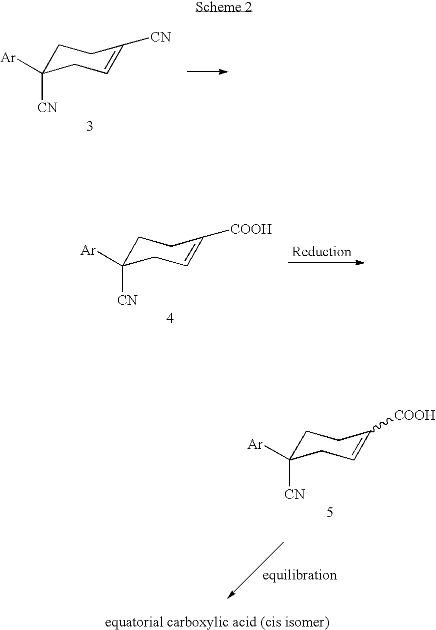
equatorial carboxylic acid (cis isomer)

Compound 4 is prepared by the hydrolysis of the α,β unsaturated nitrile 3 with base (for example 2 equivalents of Ba(OH)₂). This produces the unsaturated carboxylic acids which are stable and can be characterized. Although the catalytic hydrogenation of 3 is difficult to achieve, hydrogenation of 4 occurs readily and produces the saturated carboxylate as a mixture of cis and trans acid. This mixture can be converted to the cis isomer by equilibration of the methyl esters.

Reaction Scheme 3 illustrates how a by-product of the base hydrolysis of compound 7 in Scheme 1 can be converted to the 4-cyanocyclohexanoic acid.

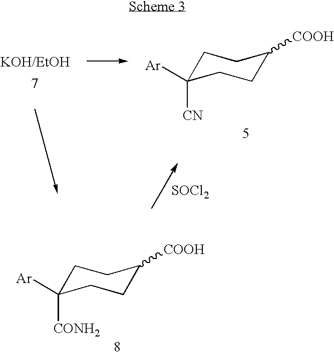

The following examples are set out to illustrate the invention. They are not intended to limit it in any way or fashion. Reference is made to the claims for what is reserved to the inventors hereunder.

Specific Exemplification

### EXAMPLE 1

### Preparation of 4-[3-(Cyclopentyloxy)-4-methoxyphenyl]-1,4-dicarbonitrile cyclohexan-1-ol: Trimethylsilyl Cyanide Method

To a 100 ml round bottom flask equipped with magnetic stirrer and a nitrogen inlet was charged 4-cyano-4-(3-cyclopentyloxy4-methoxyphenyl)cyclohexan-1-one (prepared as illustrated in, for example, U.S. Pat. No. 5,552,438) (12.50 g, 40 mmol), zinc iodide (0.35 g, 1.1 mmol) and methylene chloride (50 mL). Stirring produced a clear solution. This solution was charged with trimethylsilyl cyanide (TMSCN).(8 mL, 5.952 g, 59.9 mmol). After stirring for 1 hour under nitrogen 5 drops of the solution was quenched into dilute acid and examined by reverse phase HPLC.

The reaction was cooled to 15° C. and treated with a stream of HCl (gas) for 15 min. The reaction was evaporated on the rotovap and the resulting thick oil was suspended in ethyl acetate (150 mL) and washed with 2×50 mL 3N HCl, brine (25 mL) and finally evaporated to a residue. The residue was treated with ethanol (95%) (15 mL) and the solution cooled to 0° C. overnight. No crystallization had occurred. The solution was refluxed and treated sequentially with acetone (5 mL) and water (deionized, 15 mL). The solution was cooled to room temperature and treated with 5 mg and seeds of authentic cyanohydrin. The reaction was cooled to 0° C. and stirred for 1.5 hours. The resultant solids were filtered and washed with cold H₂O-EtOH (1:1) (10 mL).

The product was dried at 45° C. at 20 inches of mercury. The elemental analysis was satisfactory for C₂₀H₂₄N₂O₃ Theory: C, 70.58; H, 7.11; N, 8.23. Found: C, 70.66; H, 7.03; N, 8.47. The NMR in CDC₁₃ (400 MHz) was in agreement with the assigned structure (see below).

400 MHz NMR data for 4-[3-(cyclopentyloxy)-4-methoxyphenyl]-1,4-dicarbonitrile cyclohexan-1-ol

| | No. of | Multiplicity/Coupling | |
|---|---|---|---|
| ABSORPTION | protons | constant | |
| 1.59-1.62 | 2 | singlet, broad | |
| 1.75-2.0 | 6 | multiplet, broad | |
| 2.1-2.48 (incl 2.20 s) | 8 | multiplet, | |
| 2.90 | 1 | singlet | O—H |
| 3.85 | 3 | singlet | O—CH₃ |
| 4.80 | 1 | Multiplet (broad) | cyclopentyl |
| 6.85 | 1 | doublet, J = 8 Hz | Arom |
| 7.0 | 2 | Multiplet (narrow) | Arom |

Mass Spectral Data

| (Parent 340) | | | |
|---|---|---|---|
| Mass | | | |
| Absorption | Conditions | Assignment | Relative Intensity |
| 375 | Neg ion/DCI | [m + Cl⁻ | 88.5 |
| | methane | | |
| 348 | Neg ion/DCI | [375 − HCN]⁻ | 100 |
| | methane | | |
| 339 | Neg ion/DCI | [M − H]⁻ | 25.75 |
| | methane | | |
| 334 | Neg ion/DCI | | 17.1 |
| | methane | | |
| 244 | Neg ion/DCI | [375 − C5H9 − HCN]⁻ | 36.9 |
| | methane | | |
| 341 | DCI/methane | [M + H]⁺ | 19.9 |
| 314 | DCI/methane | [M + H − HCN]⁺ | 74.3 |
| 287 | DCI/methane | [M − 2 HCN] | 100.0 |
| 272 | DCI/methane | [M + 245 + C2H5] | 14.84 |
| 245 | DCI/methane | [M − C5H9 − HCN]H⁺ | 36.9 |

### EXAMPLE 2

### Alternative Preparation of 4-[3-(Cyclopentyloxy)-4-methoxyphenyl]-1,4-dicarbonitrile cyclohexan-1-ol: Sodium Cyanide Method

To a 20 mL round bottom flask equipped with magnetic stirrer and internal thermometer was added 4-cyano-4-(3-cyclopentyloxy-4-methoxyphenyl)cyclohexan-1-one (1.00 g, 3.19 mmol) and sodium cyanide (0.325 g, 6.6 mmol) in water (5 mL). The reaction was stirred and cooled to 0-5° C. and an aqueous solution of sodium bisulfite (0.625 g, in 2.5 mL water) as the temperature reached 8° C. The reaction was cooled and after 30 min. the reaction had set up as a solid. The reaction was warmed to 8-10° C., and acetone (2 mL) was added. The reaction was continued at 20° C. for 90 min. The reaction was partitioned between water (5 mL) and ethyl acetate (10 mL). The organic layer was washed with water (3×) and brine, and dried with anhydrous MgSO₄. Evaporation of the ethyl acetate gave a solid which was dried in a vacuum oven at 25° C. for 15 hours.

300 MHz NMR data for compound 2:

| | No of | Multiplicity/ | |
|---|---|---|---|
| ABSORPTION | protons | Coupling constant | |
| 1.60 | 4 | singlet, broad | |
| | (include | | |
| | solvent) | | |
| 1.75-2.0 | 6 | multiplet, broad | |
| 2.1-2.48 (incl 2.20 s) | 8 | multiplet, | |
| 2.96 | 1 | unresolved doublet | O—H |
| 3.85 | 3 | singlet | O—CH3 |
| 4.80 | 1 | Multiplet (broad) | cyclopentyl |
| 6.88 | 1 | doublet, J = 7.4 Hz | Arom |
| 7.0 | 2 | Multiplet (narrow) | Arom |

### EXAMPLE 3

### Dehydration of 4-[3-(Cyclopentyloxy)-4-methoxyphenyl]-1,4-dicarbonitrile cyclohexan-1-ol

To a 10 mL 3-necked round bottom flask equipped with magnetic stirring, a thermometer, and reflux condenser was charged with 4-[3-(cyclopentyloxy)-4-methoxyphenyl]-1,4-dicarbonitrile cyclohexan-1-ol (2 in Scheme 1) (0.50 g, 1.46 mmol), toluene (1.5 mL) and pyridine (0.60 mL). The resulting solution was cooled to 0° C. and treated with thionyl chloride (0.35 g, 2.94 mmol) in toluene (0.5 mL). A precipitate formed after 1-2 min. The reaction was heated to 80° C. and refluxed for an additional 2.0 hours. The solution was cooled and poured into a mixture of HCl and ice. The reaction was extracted with 2×15 mL of ethyl acetate. The organic phase was washed with 0.6 N HCl, 5% sodium carbonate and with brine. The organic layer was dried (MgSO4) and evaporated to a solid, i.e., compound 3. The NMR in CDC₁₃ was clean and compatible with that of the structure 4-[3-(cyclopentyloxy)-4-methoxyphenyl]-1-cyclohexene-1,4-dicarbonitrile, i.e., nitrile 3 in Scheme 1.

Elemental analysis: theoretical C₂₀H₂₂N₂O₂: C, 74.51; H, 6.88; N, 8.69. Found: C, 74.23; H, 6.98; N, 8.64.

NMR of the unsaturated nitrile in CDC₁₃ showed a diagnostic vinyl C—H at 6.67 ppm.

300 MHz NMR data

| | | Multiplicity/ | |
|---|---|---|---|
| ABSORPTION | No of protons | Coupling constant | |
| 1.55-1.67 | 3-4 | broad overlapping | |
| 1.80-2.0 | 3-4 | multiplet, broad | |
| 2.05-2.18 | 1-2 | multiplet, | |
| 2.2-2.5 | 2 | 2 groups of protons | |
| 2.6-2.9 | 2-3 | 2 groups of protons | |
| 3.84 | 3 | singlet | O—CH₃ |
| 4.80 | 1 | Multiplet (broad) | cyclopentyl |
| 6.67 | 1 | triplet (broad) | vinyl |
| | | | proton |
| 6.83-6.95 | 3 | six overlapping bands | Arom |

### EXAMPLE 4

### Alternative Method for Dehydrating 4-[3-(Cyclopentyloxy)-4-methoxyphenyl]-1,4-dicarbonitrile cyclohexan-1-ol

To a 1000 mL 3-necked round bottom flask equipped with overhead stirring, a pressure equalizing dropping funnel and a nitrogen inlet was charged crude 4-[3-(cyclopentyloxy)-4methoxyphenyl]-1,4-dicarbonitrile cyclohexan-1-ol (2) along with toluene (140 mL) and pyridine (40 mL). A clear solution was produce with stirring. The reaction was cooled to 10° C. and thionyl chloride (about 20 mL) was added at a rate to keep the reaction <15° C. The reaction was refluxed and monitored by HPLC (4.6×250 mm Beckman Ultrasphere; wavelength at 230 nm, flow rate 1.0 mL/min) until essentially all of the starting material had disappeared, approximately 2.5 hours.

The reaction was cooled to 40° C. in an ice bath and quenched with 6N HCl (100 mL) followed by addition of ethyl acetate (400 mL). The organic layer was separated and washed with two 100 mL portions of 3N HCl, 10% aqueous sodium bicarbonate (100 mL), and finally brine (100 mL). The organic layer was evaporated to constant weight and dissolved in warm isopropanol (50 mL). The solution was cooled to 0° C. and the solid was stirred at that temperature for 2 hours, and filtered and washed with cold isopropanol (10 mL). Drying in a vacuum oven at 40° C. for 16 h (10 mmol Hg) gave a solid, i.e., compound 3. The NMR in CDC₁₃ was the same as the spectrum of the product prepared in EXAMPLE 3. The melting point was 133.5-134.5° C. Elemental Analysis of a recrystallized sample: Calculated: C, 74.51; H, 6.88; N, 8.69. Found: C, 74.23; H, 6.98; N, 8.64.

### EXAMPLE 5

### Reduction of 4-[3-(Cyclopentyloxy)-4-methoxyphenyl]-1-cyclohexene-1,4-dicarbonitrile to Form 4-[3-(Cyclopentyloxy)-4-methoxyphenyl]-1-cyclohexane-1,4-dicarbonitrile

This example illustrates the preparation of compounds 7 in Scheme 1.

To a 1000 mL 3-necked round bottom flask equipped with magnetic stirring, an internal thermometer and a reflux condenser was charged with 4-[3-(cyclopentyloxy)-4-methoxyphenyl]-1-cyclohexene-1,4-dicarbonitrile, (10.00 g, 31.0 mmol) and methanol (200 mL). The solution was stirred and heated to 60° C., at which time a fine suspension resulted. The reaction was charged with magnesium turnings (4.02 g, 165 mmol, 5 equivalents) which had been activated by drying in a vacuum oven at 40-50° C. The reaction was refluxed for 3 h. The reaction mixture was cooled to <30° C. and treated with 6N HCl. The reaction solvent was evaporated on the rotovap (40° C.) to constant weight and the residue treated with ethyl acetate (200 mL). The isolated organic layer after filtration was washed with water (2×50 mL) and brine (2×15 mL). The organic layer was concentrated on a rotovap to an oil, i.e. 4-[3-(cyclopentyloxy)-4-methoxyphenyl]-1-cyclohexane-1,4-dicarbonitrile, compound 7 in Scheme 1. The product was diluted with absolute ethanol and used directly in the hydrolysis of the nitrile with potassium hydroxide.

In the same manner as above the unsaturated nitrile (2.53 g, 7.80 mmol) was reduced with Mg(0) ( 0.98 g, 40.3 mmol, 5 eq) in methanol (50 mL) at 55° C. After refluxing for 3 h, the product was isolate as an oil. this oil was used in the hydrolysis without further purification.

### EXAMPLE 6

### Base hydrolysis of 4-[3-(Cyclopentyloxy)-4-methoxyphenyl]-1-cyclohexane-1,4-dicarbonitrile

4-[3-(Cyclopentyloxy)-4-methoxyphenyl]-1-cyclohexane-1,4-dicarbonitrile (7) (2.120 g, 6.53 mmol) and ethanol (10 mL) were charged to a 100 mL 3-necked round bottom flask equipped with magnetic stirring, an internal thermometer and a reflux condenser. The solution was stirred and refluxed until clear. After removing heat the reaction was charged dropwise with a solution of KOH in water (3.4 g, 60.6 mmol in 10 mL water). This solution was then refluxed for 4 h. The reaction mixture was reduced to one-half volume on the rotovap and partitioned between ethyl acetate (50 mL) and 6 N HCl (12 mL). The organic layer was separated and washed with deionized water (2×15 mL), brine (1×15 mL), and evaporated to give a mixture of cis and trans isomers of 4-cyano-4-[3-(cyclopentyloxy)-4-methoxyphenyl]-1-cyclohexane-1-carboxylic acid as an oil.

### EXAMPLE 7

### Conversion of cis-4-(Aminocarbonyl)-4-[3-(cyclopentyloxy)-4-methoxyphenyl]cyclohexanecarboxylic acid to cis-4-Cyano-4-(3-cyclopentyloxy-4-methoxyphenyl)-r-cyclohexanecarboxylic acid

To a 5 mL 1-necked round bottom flask equipped with a magnetic stirrer, a nitrogen inlet, and an oil bath was charged the cis-4-(aminocarbonyl)-4-[3-(cyclopentyloxy)-4-methoxyphenyl]cyclohexanecarboxylic acid (0.050 g, 0.138 mmol) and toluene (0.50 mL). The flask was heated to 70° C. and the suspension treated with thionyl chloride (0.25 mL, 0.408 g, 3.43 mm, 25 eq) added in a single portion. A clear yellow solution was produced. The reaction was heated a total of 4 h at bath temperature 75° C. The reaction was cooled to 10° C. and a small aliquot was evaporated with nitrogen and examined by HPLC. The HPLC showed complete disappearance of the acid-amide and clean conversion to a mixture of the cis and trans isomers of 4-cyano-4-[3-(cyclopentyloxy)-4-methoxyphenyl]-1-cyclohexane-1-carboxylic acid.

### EXAMPLE 8

### Hydrolysis of 4-[3-(Cyclopentyloxy)-4-methoxyphenyl]-1-cyclohexane-1,4-dicarbonitrile

To a 50 mL 3-necked round bottom flask equipped with magnetic stirrer, an internal thermometer, and a reflux condenser was charged equal amounts of cis saturated nitrile (compound 7 in Scheme 1,4-[3-(cyclopentyloxy)-4-methoxyphenyl]-1-cyclohexane-1,4-dicarbonitrile) (0.5016 g, 1.54 mmol) and the corresponding trans dicarbonitrile (0.5016 g, 1.54 mmol) (total 3.08 mmol) and absolute ethanol (10 mL). The reaction was heated to reflux for 5 min, to obtain a solution. The reaction was treated slowly with a solution of NaOH (1.16 g, 29 mmol, 9.4 eq) in deionized water (10 mL). The rate of addition was adjusted to avoid precipitation of the starting nitriles. The solution was stirred and refluxed for 4-5 h. At that time the HPLC showed (80% PAR) of the desired product a cis/trans mixture of acids and a side product identified as an acid-amide, that is, the 4-position nitrile had converted to —CONH₂. The solvent was evaporated to give an oil which was treated with 6 N HCl (10 mL) and ethyl acetate (35 mL). The layers were separated and the organic layer washed with water (2×10 mL) and brine (1×5 mL) and dried (magnesium sulfate). The product (compound 5 and its 4-position —CONH₂ analog cis-4-(aminocarbonyl)-4-[3-(cyclopentyloxy)-4-methoxyphenyl]-1-cyclohexane-1-carboxylic acid) was concentrated by evaporation at reduced pressure.

This crude product was placed in a 50 mL 3-neck flask equipped with a magnetic stirrer, nitrogen inlet, and a thermometer. Toluene (9.0 mL) was added and then thionyl chloride (1.0 mL, 1.6 g) and the reaction heated at 70-75° C. The reaction was judged to be complete after 3 h. The reaction was evaporated on the rotovap to 4 mL, and transferred to a separatory funned with ethyl acetate (30 mL). The top layer was washed with water (2×5.5 mL), 3 N HCl (1×5 mL), water (1×5 mL) and brine (1×5 mL) before drying with magnesium sulfate. The resulting organic layer was evaporated to constant weight (1.15 g) and dissolved in boiling ethyl acetate and treated with hexane. The product was further treated by using a base-acid treatment of the combined aqueous layers, followed by extraction into ethyl acetate. Evaporating the ethyl acetate gave a residue which was dissolved then re-dissolved in ethyl acetate (5 mL) and hexane (4 mL). Product, a mixture of cis and trans 4-cyanocyclohexanoic acid, was crystallized from this solvent system.

### EXAMPLE 9

### Alternative Process for Hydrolyzing 4-[3-(Cyclopentyloxy)-4-methoxyphenyl]-1-cyclohexene-1,4-dicarbonitrile

To a 100 mL 3-necked round bottom flask equipped with magnetic stirrer, an internal thermometer, and a reflux condenser was charged the cis/trans saturated nitriles (compound 7, 4-[3-(cyclopentyloxy)-4-methoxyphenyl]-1-cyclohexene-1,4-dicarbonitrile) (2.12 g, 6.54 mmol) and absolute ethanol (10 mL). The reaction was heated to reflux; a solution was obtained. The reaction was treated slowly with a solution of KOH (3.4 g, 60.6 mmol, 9.3 eq) in deionized water (10 mL). The addition was done dropwise over 10 min. The solution was stirred and refluxed for 4-5 h. At that time the HPLC showed the absence of starting material and the presence of 4-cyano-4-[3-(cyclopentyloxy)-4-methoxyphenyl]-1-cyclohexane-1-carboxylic acid and the side product, the 4-carboxamide 8 (15% PAR). The reaction was evaporated to 1/2 volume and treated with 6 N HCl (12 mL) and ethyl acetate (50 mL). The layers were separated and the organic layer was washed with water (2×15 mL) and brine (1×10 mL) and dried (magnesium sulfate). A crude product was obtained by evaporation at reduced pressure.

The crude product obtained as per the previous paragraph was placed in a 100 mL 3-neck flask equipped with a magnetic stirrer, distillation head, and a thermometer. Toluene (15.0 mL) was added and then distilled to a volume of 6 mL. Then fresh toluene (15 mL) and thionyl chloride (2.0 mL, 3.2 g) was added and the reaction heated at 70-75° C. After 1.5 h some unreacted starting material had not gone into solution, so the reaction was charged with thionyl chloride (1 mL) and toluene (5 mL). After 4 h more the reaction was judged to be complete (HPLC assay). The mixture was filtered and the filtrate evaporated to constant weight.

The filtrate (2.71 g) from the preceding paragraph was charged to a 100 mL round bottom flask equipped with a magnetic stirrer along with anhydrous tetrahydrofuran (15 mL). To this well stirred solution was added 20% aqueous NaOH (3 mL) after which the mixture was stirred at room temperature for 1 h. The resulting reaction was concentrated on the rotovap, treated with ethyl acetate (50 mL) and 6 N HCl (10 mL) and the layers separated. The organic layer was isolated and washed with water (2×10 mL) brine (1×5 mL) and dried over magnesium sulfate. Removal of the volatiles left a thick oil which was dried at 50° C. for 16 h at 20 mm of Hg. The resulting semisolid (2.00 g) was dissolved in boiling ethyl acetate (10 mL) and then hot hexane (8 mL). The reaction was slowly cooled to −3° C. and stirred for 2 h. A thick solid was present. The solid was collected and washed with a cold ethyl acetate/hexane mixture. After drying in a vacuum oven at 50° C. (1 mm of Hg), a white solid was obtained (compound 5 in Scheme 1).

300 MHz NMR data: c-4-cyan-4-(3-cyclopentyloxy-4-methoxyphenyl)-r-cyclohexanecarboxylic acid in CDC₁₃

| | No of | Multiplicity/ | |
|---|---|---|---|
| ABSORPTION | protons | Coupling constant | |
| 1.64-1.7 | 2 | singlet, broad | |
| 1.86-1.98 | 9-10 | multiplet, broad | |
| 2.28 | 4 | triplet (distorted) | |
| 2.45 | 1 | multiplet | |
| 3.89 | 3 | singlet | O—CH₃ |
| 4.84 | 1 | septuplet | cyclopentyl |
| 6.89 | 1 | doublet, J = 8.4 Hz | Arom |
| 7.01, 7.02, 7.041, 7.048 | 2 | Multiplet (narrow) | Arom |

Carbon 13 NMR (90 MHz, CDCl₃): 180.22, 149.10, 147.84, 132.78, 122.15, 117.34, 112.97, 111.96, 80.74, (solvent 76 t), 56.07, 42.98, 41.63, 36.44, 32.77, 25.88, 24.10. [Italicized signals are C—H's and C—H3's].
Alternate Synthesis

### EXAMPLE A

### Hydrolysis of 4-[3-(Cyclopentyloxy)-4-methoxyphenyl]-1-cyclohexene-1,4-dicarbonitrile

To a 100 mL 3-necked round bottom flask equipped with a magnetic stirrer, a reflux condenser, and a nitrogen inlet was charged with 4-[3-(cyclopentyloxy)-4-methoxyphenyl]-1-cyclohexene-1,4-dicarbonitrile nitrile, compound 3 in Scheme 1, (2.00 g, 6.16 mmol) and absolute ethanol (25 mL). The reaction was refluxed for 5 minutes; a clear solution resulted. To this was added a suspension of Ba(OH)₂.8H₂O in water (6.00 g, 19 mmol, in 25 mL of distilled water). The solution was refluxed for 3.5 hrs and cooled to room temperature. The reaction was acidified with 3 N HCl and extracted with ethyl acetate (1×) and t-butylmethyl ether (1×). The organic layers were combined and washed with water (2×15 mL) and brine. After drying with magnesium sulfate the unsaturated acid 4-cyano-4-[3-(cyclopentyloxy)-4methoxyphenyl]-1-cyclohexene-1-carboxylic acid (compound 5 in Scheme 1) was isolated as an oil by evaporation of the solvents on the rotovap. This oil also contained an impurity identified as the unsaturated acid with a 4-carboxamide (formed by concomitant hydrolyis of the 4-position nitrile). The product was purified by preparative reverse phase liquid chromatography. The product, compound 4 in Scheme 1, weighed 0.450 g. The unsaturated acid with a 4-carboxamide group was isolated and identified as indicated below.

IR FT-IR (KBr): 3300 (s, O—H st), 2235 (CN stretch), 1689 (C═O stretch of unsat carboxylic acid); 1650 (C═C stretch), 1517 (C═C stretch), 1434, 1419, (C—H deform), 1258 and 1145 (C—O stretch).

The NMR (360 mHz) had a signal diagnostic for the vinyl proton at 7.0 ppm (C6D6) and 7.16 ppm (CDCl₃).

Proton NMR (360 MHz) c-4-cyano-4-(3-cyclopentyloxy-4-methoxyphenyl)-r-cyclohexanecarboxylic acid taken in C6D6.

| | No of | Multiplicity/ | |
|---|---|---|---|
| ABSORPTION | protons | Coupling constant | |
| 1.41-2.09 | 10 | 4 clusters (broad) | |
| 2.15-2.27 | 1.5 | doublet | allylic protons |
| 2.38-2.52 | 1.5 | doublet | allylic protons |
| 2.80 | 1 | Very broad | allylic proton |
| 3.50 | 3 | singlet | O—CH₃ |
| 4.72 | 1 | septuplet | cyclopentyl |
| 6.63 | 1 | doublet, J = 8.4 Hz | arom (α to OCH₃) |
| 6.82 | 1 | doublet of doublets, | arom |
| | | (J = 8.4 + meta | |
| | | coupling, J = 2) | |
| 7.0 | 1 | broad singlet | vinyl proton |
| 7.1 | 1 | doublet (meta, J = 2) | Isolated aryl C—H |

C-13 NMR of unsaturated carboxylic acid (CHCL₃, 90 HZ).): 170.7, 149.9, 147.9, 137.36, 131.5, 129.7, 122.3, 117.5, 112.7, 111.9, 80.69, 56.1, 39.7, 33.0, 32.8, 32.6, 24.1, 22.25.

### EXAMPLE B

Transfer hydrogenation of 4-Cyano-4-[3-(cyclopentyloxy)-4-methoxyphenyl]-1-cyclohexene-1-carboxylic acid (4) to c-4-Cyano-4-(3-cyclopentyloxy-4-methoxyphenyl)-r-cyclohexanecarboxylic acid (5)

To a 5 mL 2-necked round bottom flask equipped with a magnetic stirrer and a nitrogen inlet was charged 4-cyano-4-[3-(cyclopentyloxy)-4-methoxyphenyl]-1-cyclohexene-1-carboxylic acid (4 in Scheme 1) ( 0.021 g, 0.06 mmol) and dimethyl formamide (DMF) (0.5 mL). Ammonium formate (0.050 g) was added to the pot and hydrogenation catalyst (10% Pd/C; Aldrich 20569-9). The reaction was stirred at 20° C. for 20 h. The reaction was filtered and the dimethyl formamide removed under high vacuum. The product was extracted into ethyl acetate (2×4 ml). The organic layers were washed with water (2×4 mL) and brine (1×5 mL). Evaporation gave an oil which had an NMR the same as reference material of the cis form of the desired acid, 4-cyano-4-[3-(cyclopentyloxy)-4-methoxyphenyl]-1-cyclohexane-1-carboxylic acid, 5 in Scheme 1.

1. A process for reducing an α,β-unsaturated cyclohexene carboxylic acid, which process comprises catalytically reducing the double bond using a heavy metal catalyst and an ammonium bicarbonate.

2. The process of claim 1 wherein the reducing agent is 10% Pd/C and the ammonium carbonate is ammonium formate.

3. A process for preparing a compound of formula (I) 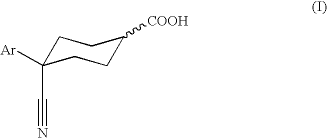
where Ar is an aromatic group,
wherein the process comprises reducing a cyclohexene of formula (A).

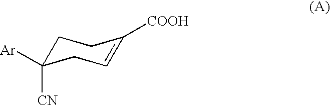

4. The process according to claim 2 wherein, in the compound of formula (A), Ar is an aromatic group of Formula (i) 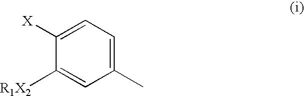
wherein:
R₁ is —(CR₄R₅)ᵣR₆ wherein the alkyl moiety may be optionally substituted with one or more halogens;
R₂ is —CH₃ or —CH₂CH₃ optionally substituted by 1 or more halogens;
r is 0 to 6;
R₄ and R₅ are independently selected from hydrogen or a C₁₋₂ alkyl;
R₆ is hydrogen, methyl, hydroxyl, aryl, halo substituted aryl, aryloxyC₁₋₃ alkyl, halo substituted aryloxyC₁₋₃ alkyl, indanyl, indenyl, C₇₋₁₁ polycycloalkyl, tetrahydrofuranyl, furanyl, tetrahydropyranyl, pyranyl, tetrahydrothienyl, thienyl, tetrahydrothiopyranyl, thiopyranyl, C₃₋₆ cycloalkyl, or a C₄₋₆ cycloalkyl containing one or two unsaturated bonds, wherein the cycloalkyl and heterocyclic moieties may be optionally substituted by 1 to 3 methyl groups or one ethyl group;
X is YR₂; and
Y is O or S(O)ₘ where m is 0, 1 or 2.

5. The process of claim 4 wherein, in formula (1), R₁ is —CH₂-cyclopropyl, cyclopentyl, 3-hydroxycyclopentyl, methyl or CF₂H; R₂ is methyl, —CHF₂ or —CH₃; Y is oxygen; and X₂ is oxygen.

6. The process according to claim 5 wherein the compound of formula (A) is 4-cyano-4-[3-(cyclopentyloxy)-4-methoxyphenyl]-1-cyclohexene-1-carboxylic acid.

7. The process of claim 3 wherein the double bond is reduced using catalytic hydrogenation using a heavy metal catalyst and an ammonium carbonate.

8. The process of claim 3 wherein the reducing agent is 10% Pd/C and ammonium formate.

9. A process for preparing a compound of formula (I) 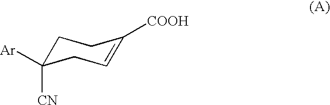
wherein Ar is the group formula (i) 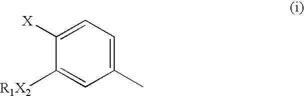
wherein:
R₁ is —(CR₄R₅)ᵣR₆ wherein the alkyl moiety may be optionally substituted with one or more halogens;
R₂ is —CH₃ or —CH₂CH₃ optionally substituted by 1 or more halogens;
r is 0 to 6;
R₄ and R₅ are independently selected from hydrogen or a C₁₋₂ alkyl;
R₆ is hydrogen, methyl, hydroxyl, aryl, halo substituted aryl, aryloxyC₁₋₃ alkyl, halo substituted aryloxyC₁₋₃ alkyl, indanyl, indenyl, C₇₋₁₁ polycycloalkyl, tetrahydrofuranyl, furanyl, tetrahydropyranyl, pyranyl, tetrahydrothienyl, thienyl, tetrahydrothiopyranyl, thiopyranyl, C₃₋₆ cycloalkyl, or a C₄₋₆ cycloalkyl containing one or two unsaturated bonds, wherein the cycloalkyl and heterocyclic moieties may be optionally substituted by 1 to 3 methyl groups or one ethyl group;
X is YR₂; and
Y is O or S(O)m where m is 0, 1 or 2;
which process comprises:
hydrolyzing an α,β-unsaturated cyclohexene dicarbonitrile of formula (B); 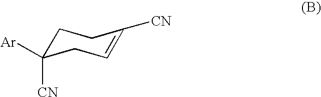
wherein the Ar group is the same as in formula (A).

10. The process of claim 9 wherein, in formula (1), R₁ is —CH₂-cyclopropyl, cyclopentyl, 3-hydroxycyclopentyl, methyl or CF₂H; R₂ is methyl, —CHF₂ or —CH₃; Y is oxygen; and X₂ is oxygen.

11. The process according to claim 10 wherein the compound of formula (A) is 4-cyano-4-[3-(cyclopentyloxy)-4-methoxyphenyl]-1-cyclohexene-1-carboxylic acid.

12. The process according to claim 9 wherein the hydrolysis is carried out using thionyl chloride.

13. A process for preparing a α,β-unsaturated cyclohexene dicarbonitrile of formula (B) 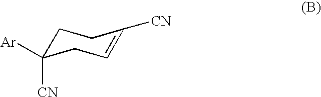
wherein Ar is the group formula (i) 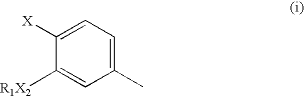
wherein:
R₁ is —(CR₄R₅)ᵣR₆ wherein the alkyl moiety may be optionally substituted with one or more halogens;
R₂ is —CH₃ or —CH₂CH₃ optionally substituted by 1 or more halogens;
r is 0 to 6;
R₄ and R₅ are independently selected from hydrogen or a C₁₋₂ alkyl;
R₆ is hydrogen, methyl, hydroxyl, aryl, halo substituted aryl, aryloxyC₁₋₃ alkyl, halo substituted aryloxyC₁₋₃ alkyl, indanyl, indenyl, C₇₋₁₁ polycycloalkyl, tetrahydrofuranyl, furanyl, tetrahydropyranyl, pyranyl, tetrahydrothienyl, thienyl, tetrahydrothiopyranyl, thiopyranyl, C₃₋₆ cycloalkyl, or a C₄₋₆ cycloalkyl containing one or two unsaturated bonds, wherein the cycloalkyl and heterocyclic moieties may be optionally substituted by 1 to 3 methyl groups or one ethyl group;
X is YR₂; and
Y is O or S(O)m where m is 0, 1 or 2;
which process comprises dehydrating a 1,4-dicarbonitrile cyclohexan-1-ol of formula (C) using a base 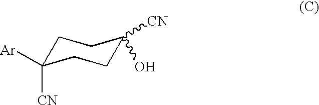
wherein Ar is the same as defined for formula (B).

14. The process of claim 13 wherein, in the Ar group, R₁ is —CH₂-cyclopropyl, cyclopentyl, 3-hydroxycyclopentyl, methyl or CF₂H; R₂ is methyl, —CHF₂ or —CH₃; Y is oxygen; and X₂ is oxygen.

15. The process of claim 13 wherein formula (i) is 3-(cyclopentyloxy)-4-methoxyphenyl-

16. The process of claim 13 wherein the base used to dehydrate formula (C) is Ba(OH)₂.

17. A process for preparing a compound of formula (C) as described in claim 13 by the cyanohydrin homologation of cyclohexanone (D) as described herein 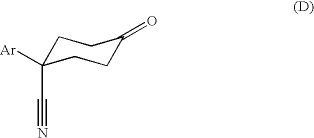
wherein Ar is the aromatic group of formula (i) as defined in claim 13.

18. The process of claim 17 wherein the homologation is carried out using trialkylsilyl cyanide.

19. The process of claim 17 wherein the cyanide is trimethylsilyl cyanide and the compound of formula (i) is 3-cyclopentyloxy-4-methoxyphenyl.

20. The process of claim 17 wherein the homologation is carried using an alkali metal cyanide salt.

21. The process of claim 19 wherein the cyanide salt is NaCN and the compound of formula (i) is 3-(cyclopentyloxy)-4-methoxyphenyl.
